(19) United States
(12) Patent Application Publication
(10) Pub. No.: US 2012/0301866 A1
(43) Pub. Date: Nov. 29, 2012

(54) LIQUID FORMULATION HAVING DISSOLVED GASES USEFUL FOR PRESERVING BIOLOGICAL MATERIAL

(75) Inventors: Andrew Martin, Versailles (FR); Marc Lemaire, Paris (FR); Jan Pype, Herne (BE); Chui Fung Chong, Paris (FR)

(73) Assignee: L'Air Liquide, Société Anonyme pour I'Etude et I'Exploitation des Procédés Georges Claude, Paris (FR)

(21) Appl. No.: 13/578,029

(22) PCT Filed: Jan. 21, 2011
(86) PCT No.: PCT/FR2011/050112

§ 371 Date: Aug. 9, 2012

(30) Foreign Application Priority Data
Feb. 18, 2010 (FR) 1051147

(51) Int. Cl. A01N 1/02; C12N 5/071

(52) U.S. Cl. 435/1.1; 435/374; 435/366

(57) ABSTRACT

The invention relates to a liquid formulation including a liquid solution and at least one gas selected from xenon, argon, hydrogen, H₂S, helium, krypton, neon, radon or CO, said gas being dissolved in said liquid solution, for the use thereof as a preservative solution for preserving biological material, in particular cells, tissue and biological organs, in particular an organ selected from the heart, the kidney, the liver, the pancreas and the intestines. The gas is preferably argon.

### CROSS REFERENCE TO RELATED APPLICATIONS

This application is a 371 of International PCT Application PCT/FR2011/050112, filed Jan. 21, 2011, which claims priority to French Application 1051147, filed Feb. 18, 2010, the entire contents of which are incorporated herein by reference.

### BACKGROUND

The present invention relates to a liquid formulation which contains one or more dissolved gases, more particularly argon, for preserving biological materials, such as organs, tissues or cells, in particular biological materials for transplantation, and to a method for preserving said biological materials using a cold solution, saturated with a gas or gases, and stored in a chamber under a gaseous atmosphere which comprises the same gas(es).

The storage, in particular for transplantation purposes, of an organ is often limited by injuries caused by ischemic reperfusion in said organs. Under ischemic conditions, adenosine triphosphate (ATP) is exhausted and the resulting lack of oxygen converts the aerobic metabolism into an anaerobic metabolism. The subsequent events may be intracellular acidosis, cell edema, the enzymatic cascades of an inflammation and apoptosis. During the reperfusion of an ischemic organ, tissue or cell, i.e. after a transplantation, reactive oxygen species, nitric oxide (NO) and pro-inflammatory cytokines are released concomitantly with the expression of adhesion molecules. This leads first to the mobilization and the trapping of leukocytes in the transplanted organ and, subsequently, to certain dysfunctions of the transplanted organs, as taught by S. Reddy et al., “Liver transplantation from non-heart-beating donors: current status and future prospects” Liver Transpl 2004; 10 (10):1223-32.

Cold preservation, at approximately 4° C., of the organs or tissues slows down the metabolism and limits the effects of ischemia, even though considerable metabolic activity nevertheless exists at only approximately 1° C., as taught by P. A. Clavien et al., “Preservation and reperfusion injuries in liver allografts. An overview and synthesis of current studies.” Transplantation 1992; 53 (5):957-78.

The addition of preserving solutions such as the University of Wisconsin solution prevents the cells from swelling during ischemic cold storage. These solutions increase the antioxidant capacity of the organs (glutathione) and stimulate the generation of high-energy phosphate (adenosine) at the time of reperfusion. Although this method for preserving organs is effective, some organs, for example 5 to 15% of livers and 20 to 30% of kidneys, do not function well at the time of transplantation, as described by J. H. Southard et al., “Organ preservation.” Ann. Rev. Med., 1995; 46:235-47.

Thus, static cold storage in existing solutions is inadequate for ensuring that an organ functions after transplantation, in particular from non-heart-beating donors.

In addition, in machine perfusion systems, the organ is attached to a pump by the artery, which continually pumps a cold preserving solution through the organ. The solution provides nutrients and sometimes oxygen, removes toxic metabolites and reduces lactic acid accumulation. These systems can also have the ability to monitor the flow rate, the pressure and the internal resistance of the organ and to evaluate its viability, as explained by M. L. Henry, “Pulsatile preservation in renal transplantation”; Transplant. Proc. 1997; 29 (8):3575-6.

A crucial question relating to the application of hypothermic machine perfusion in preserving the liver is the critical balance between perfusion pressure and the occurrence of endothelial injuries, as taught by N. A. van der PA 't Hart et al.; “Hypothermic machine perfusion of the liver and the critical balance between perfusion pressures and endothelial injury.” Transplant Proc 2005; 37 (1):332-4.

In addition, hypothermic machine perfusion requires continuous monitoring and correction of the chemical compositions and also of the pressure and flow rate in order to be optimal. Thus, the process requires a lot of time and labor and, consequently, is expensive.

As a general rule, organ perfusion requires considerable expertise and the results can be very different from one perfusionist to the other.

Another problem with organ preservation that is observed with the current kidney perfusion solutions is the rapid oxidation of glutathione, which is a key component of the current kidney perfusion solutions that serves as an antioxidant, which is reflected by a reduction in the elimination of free radicals by oxidation. This will be detrimental to the quality of the organ preservation and will lead to poor results after transplantation.

It has already been proposed to use hyperbaric atmosphere for preserving organs. More specifically, gases at high pressure have been applied in order to increase the dissolved oxygen saturation concentration.

However, because of the complexity of the apparatus required and the potential for damage to the organ during the compression or expansion of the gases, as the hyperbaric chamber is filled and opened later, this solution is not considered to be satisfactory.

Consequently, the problem to be solved is that of providing an effective method for preserving organs, in particular organs that will later be transplanted.

### SUMMARY

The solution of the present invention is a liquid formulation comprising a liquid solution and at least one gas selected from xenon, argon, hydrogen, H₂S, helium, krypton, neon, radon or CO, said gas being dissolved in said liquid solution, for use as a preserving solution for preserving a biological material, the concentration of gas dissolved in the liquid formulation, expressed as a molar fraction, being from 0.1×10⁻⁴ to 4×10⁻⁴.

### BRIEF DESCRIPTION OF THE DRAWINGS

In order to demonstrate the effectiveness of a liquid formulation in accordance with the present invention, comparative studies were carried out and the results obtained are given in the following examples and are illustrated in the figures, among which:

FIGS. 1A and 1B represent curves for creatinine clearance compared with the preoperative value on day 14 after transplantation (FIG. 1A) and the postoperative change on days 7 and 14 (FIG. 1B),

FIGS. 2A and 2B represent curves for urinary albumin compared with the preoperative value on day 14 after transplantation (FIG. 2A) and the postoperative change on days 7 and 14 (FIG. 2B),

FIGS. 3A to 3D show histological observations of the transverse section of rat kidneys on day 14 after transplantation, and

FIGS. 4A to 4E show immunohistochemical results of rat kidneys on day 14 after transplantation.

### DESCRIPTION OF PREFERRED EMBODIMENTS

In the context of the invention, the term “molar fraction” refers to the number of moles of gas considered divided by the total number of moles of all the substances, including the water, present in the liquid solution. As appropriate, the liquid formulation of the invention can comprise one or more of the following characteristics:
the gas is argon;
said biological material is selected from biological cells, tissues and organs;
said biological material is a human material;
said biological material is an organ selected from the heart, the kidney, the liver, the pancreas and the intestines;
said biological material is a biological tissue or biological cells selected from bones, the bone marrow, tendons, the cornea, the heart valves, the veins, the arms, stem cells and the skin;
said liquid solution comprises water and at least one other substance selected from buffers, colloidal substances, impermeability agents, buffers, electrolytes, ROS (reactive oxygen species) eliminators and adenosine;
it comprises a dissolved gas concentration of from 0.1×10⁻⁴ to 0.5×10⁻⁴, preferably from 0.3×10⁻⁴ to 0.5×10⁻⁴, expressed as a molar fraction;
said biological material is a human organ to be transplanted.

The invention also relates to a method for preserving a biological material, in which the biological material to be preserved is brought into contact with a liquid formulation saturated with one or more gas(es) according to the invention. As appropriate, the method of the invention can comprise one or more of the following characteristics:
the liquid formulation is at a temperature of between 2° C. and 37° C., preferably less than 15° C., more preferably less than 10° C., typically about from 3 to 6° C.;
said biological material is selected from the heart, the kidney, the liver, the pancreas and the intestines;
the biological material is placed in a receptacle, such as a container, and it is at least partially immersed in the liquid formulation, preferably totally immersed in the solution;
the receptacle comprises the liquid formulation, the biological material to be preserved and a gaseous atmosphere, said gaseous atmosphere comprising the gas(es) dissolved in the liquid formulation;
the gas is advantageously argon.

In other words, the invention also relates to a method for preserving a biological material, in which the biological material to be preserved is brought into contact with a liquid formulation comprising a liquid solution and at least one gas dissolved in said liquid solution, in which:
the biological material is an organ selected from the heart, the kidney, the liver, the pancreas and the intestines,
the gas is selected from xenon, argon, hydrogen, H₂S, helium, krypton, neon, radon and CO, and
the liquid solution is saturated with gas and comprises a dissolved gas concentration of from 0.1×10⁻⁴ to 4×10⁻⁴ expressed as a molar fraction of the number of moles of gas divided by the number of total moles of all the substances in the liquid solution.

Generally, the present invention therefore proposes dissolving protective gases or protective gas mixtures in a cold preservation solution for organs in order to obtain a gas-saturated liquid formulation which can be used to improve the survival of biological materials, such as organs, tissues and cells, during preservation and after transplantation of said biological materials.

According to the invention, the gases that can be used are selected from xenon, argon, hydrogen, H₂S, helium, krypton, neon, radon and CO, since these gases have cytoprotective effects.

Preferably, the gas to be dissolved in the liquid formulation is argon.

Concentration ranges for the argon and the xenon in organ preservation solutions according to the present invention are given in table 1 (measurements carried out at 5° C.).

TABLE 1

| Concentration in | Minimum | Maximum | Preferred range |
|---|---|---|---|
| liquid solution (*) | (at 1 atm) | (at 1 atm) | (at 1 atm) |
| Argon | 0.1 × 10⁻⁴ | 4 × 10⁻⁴ | 0.3 × 10⁻⁴ to 0.5 × 10⁻⁴ |
| Xenon | 0.3 × 10⁻⁴ | 1.4 × 10⁻³ | 1.2 × 10⁻⁴ to 1.6 × 10⁻⁴ |

(*): expressed as a molar fraction (= the moles of gas divided by the total moles of all the substances, including the water, in the liquid solution).

When a liquid formulation according to the present invention is used to preserve biological materials, their survival and their viability after transplantation are increased through a reduction in reactive oxygen species (ROSs) which damage the organ (heart, kidney, liver, pancreas and intestines), the tissues (bone, bone marrow, tendons, cornea, heart valves, veins, arms, stem cells and skin) or the individual cells taken.

Furthermore, the gases also improve the tolerance to hypoxia of the biological materials during the ischemic period.

The liquid formulation saturated with gas, such as argon, can be placed in a receptacle and the biological material to be preserved is immersed in said liquid formulation in such a way that it is protected by the action of the fluid and of the gas molecules contained in said fluid.

Preferably, the temperature of the formulation is maintained between 2 and 10° C., preferably approximately 3 to 6° C. The receptacle can be stored in a refrigeration unit.

According to the present invention, the gas must be dissolved in a liquid solution which comprises water and other substances, such as colloidal substances, for example HES or PEG-35; impermeability agents, for example citrate, glucose, histidine, lactobionate, mannitol, raffinose or sucrose; buffers, for example KH₂PO₄; electrolytes, for example Na, K or Cl; ROS eliminators, for example glutathione; or additives, for example adenosine.

In fact, many liquid solutions suitable for preserving organs are available on the market. For example, some examples of organ preservation solutions in which a gas may be dissolved in order to prepare a liquid formulation in accordance with the present invention, and also the compositions thereof, are given in table 2 (Maathuis et al. “Perspectives in organ preservation.” Transplantation 2007; 83: 1289-1298).

TABLE 2

| | | | | | | |
|---|---|---|---|---|---|---|
| EC | HOC | PBS | UW | HTK | CEL | IGL-1 |

Colloids (g/l)

| | | | | | | | |
|---|---|---|---|---|---|---|---|
| HES | — | — | — | 50 | — | — | — |
| PEG-35 | — | — | — | — | — | — | 1 |

Impermeability agents (mM)

| | | | | | | | |
|---|---|---|---|---|---|---|---|
| Citrate | — | 80 | — | — | — | — | — |
| Glucose | 195 | — | — | — | — | — | — |
| Histidine | — | — | — | — | 198 | 30 | — |
| Lactobionate | — | — | — | 100 | — | 80 | 100 |
| Mannitol | — | 185 | — | — | 38 | 60 | — |
| Raffinose | — | — | — | 30 | — | — | 30 |
| Sucrose | — | — | 140 | — | — | — | — |

Buffers (mM)

| | | | | | | | |
|---|---|---|---|---|---|---|---|
| Citrate | — | 80 | — | — | — | — | — |
| Histidine | — | — | — | — | 198 | 30 | — |
| K₂HPO₄ | 15 | — | — | — | — | — | — |
| KH₂PO₄ | 43 | — | — | 25 | — | — | 25 |
| NaHCO₃ | 10 | — | — | — | — | — | — |
| NaH₂PO₄ | — | — | 13 | — | — | — | — |
| Na₂HPO₄ | — | — | 56 | — | — | — | — |

Electrolytes (mM)

| | | | | | | | |
|---|---|---|---|---|---|---|---|
| Calcium | — | — | — | — | 0.0015 | 0.25 | 0.5 |
| Chloride | 15 | — | — | 20 | 32 | 42 | — |
| Magnesium | — | — | — | — | 4 | 13 | — |
| Magnesium | — | 40 | — | 5 | — | — | 5 |
| sulfate | | | | | | | |
| Potassium | 115 | 79 | — | 120 | 9 | 15 | 25 |
| Sodium | 10 | 84 | 125 | 25 | 15 | 100 | 120 |

ROS eliminators (mM)

| | | | | | | | |
|---|---|---|---|---|---|---|---|
| Allopurinol | — | — | — | 1 | — | — | 1 |
| Glutathione | — | — | — | 3 | — | 3 | 3 |
| Mannitol | — | 185 | — | — | 38 | 60 | — |
| Tryptophan | — | — | — | — | 2 | — | — |

Additives (mM)

| | | | | | | | |
|---|---|---|---|---|---|---|---|
| Adenosine | — | — | — | 5 | — | — | 5 |
| Glutamic | — | — | — | — | — | 20 | — |
| acid | | | | | | | |
| Keto- | — | — | — | — | 1 | — | — |
| glutarate | | | | | | | |

Key:
EC: EuroCollins;
HOC: hypertonic solution of citrate/Marshalls;
PBS: phosphate buffered sucrose;
UW: University of Wisconsin cold storage solution;
CEL: Celsior;
HTK: histidine-tryptophan-ketoglutarate;
IGL-1: Institut George Lopez;
HES: hydroxyethyl starch;
PEG-35: polyethylene glycol with an average molecular weight of 35 kDa;
ROS: reactive oxygen species.

In accordance with the present invention, argon gas was dissolved in a liquid organ preservation solution available on the market, i.e. the CELSIOR solution, the composition of which is given in table 1, in order to obtain a liquid formulation in accordance with the present invention.

For the purposes of comparison, other gases, i.e. xenon, air and nitrogen, were dissolved in the same type of liquid solution.

The liquid formulation comprising the solution with the dissolved gas was always stored and maintained at a temperature less than or equal to approximately 10° C.

In order to evaluate the renal graft preservation properties of argon or other gases, rat kidneys were removed and stored in a gas-saturated liquid solution according to the present invention.

After six hours of organ preservation at a temperature of 4° C. and at atmospheric pressure, the rat kidneys are transplanted and the survival time, renal function and reperfusion/ischemia injuries are studied by means of biochemical and histological analyses.

On days 0 (pretransplantation), 7 and 14 (after transplantation), biochemical analyses, i.e. creatinine clearance and urinary albumin, are carried out in the manner described by M. Yin et al., in “Carolina rinse solution minimizes kidney injury and improves graft function and survival after prolonged cold ischemia.” Transplantation 2002; 73:1410-1420).

On day 14 after transplantation, the kidneys are removed, weighed and cut into blocks. The kidneys are then fixed with a buffered 4% formaldehyde infusion for 24 h and they are embedded in paraffin. Five-micrometer sections are obtained from the blocks and stained with hematoxylin-eosin-safran in order to examine them by optical microscopy.

Immunodetections were carried out on serial cryostat sections 5 μm thick, using certain specific antibodies, i.e. anti-active caspase-3 and anti-CD10. The kidneys of a normal rat are used as control.

After having been rinsed, the sections are incubated for 30 minutes with biotinylated secondary antibodies and they are then visualized using avidin-biotin peroxidase.

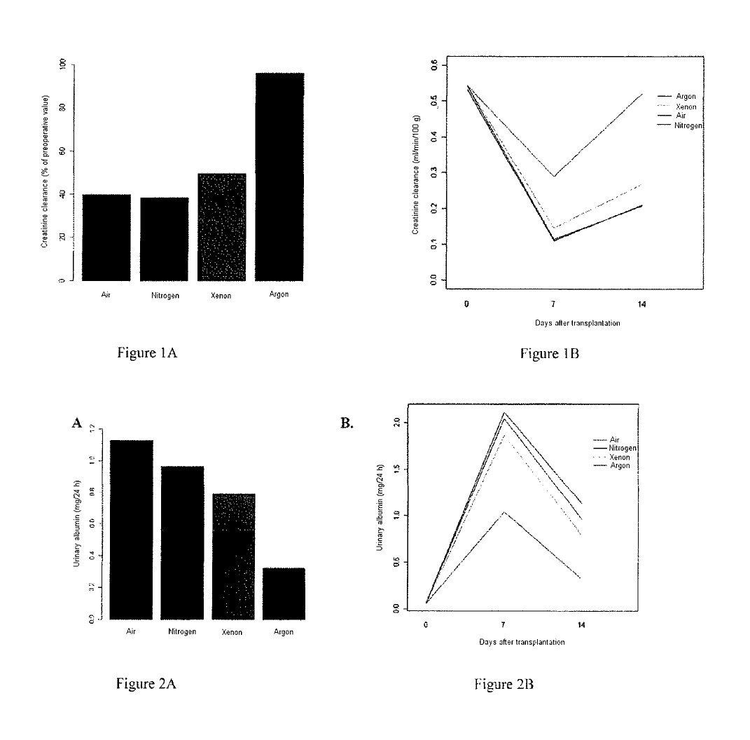

Creatinine clearance is a parameter that is used to evaluate renal function. A high clearance corresponds to good renal function, while the presence of albumin in the urine (urinary albumin) indicates renal damage since, normally, there is no albumin in the urine when the kidney is normal. Moreover, as shown in FIG. 1A, which represents the creatinine clearance (expressed in the form of percentage clearance on day 14 compared with the preimplantation value) for the four experimental groups, and FIG. 1B, which represents the change, as a function of time, in the creatinine clearance, i.e. on day 0 (preoperative value), and on day 7 and on day 14 after transplantation, argon gives the best results with regard to maintaining creatinine clearance compared with the effects observed with the other gases.

Similarly, FIG. 2A shows the level of albumin in the urine on day 14 after transplantation for the four experimental groups, while FIG. 2B shows the change in the level of albumin in the urine on day 0 (preoperative value), and on day 7 and day 14 after transplantation.

Here again, the use of argon dissolved in a liquid solution in accordance with the present invention for preserving the kidneys before transplantation gives the best results compared with the use of the other gases that were tested. Indeed, with argon, the level of albumin in the urine of rats, after the transplantation of a kidney, is much lower than the levels of albumin (urinary albumin) obtained with the other transplanted kidneys that were in contact with liquid solutions saturated with xenon, with air or with nitrogen.

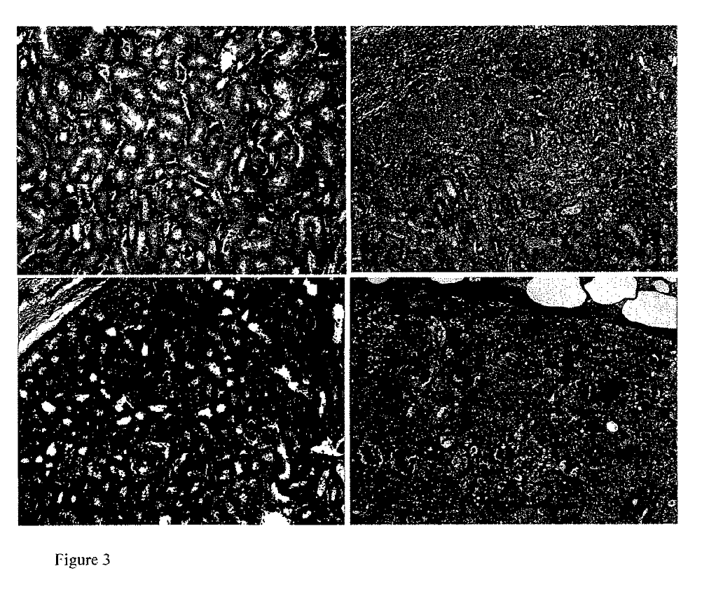

In the two cases (FIGS. 2 and 3), xenon shows a positive effect, i.e. an effect greater than that which was obtained with the controls, but which is less than that obtained with argon, which is undeniably the most effective gas that was tested.

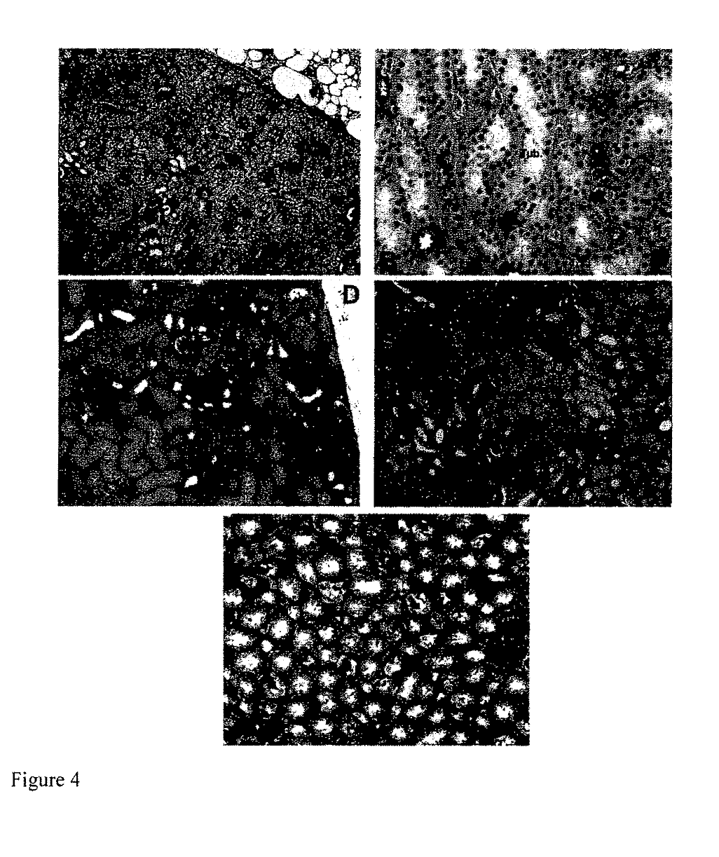

In addition, after the histological and immunohistochemical observations, it was demonstrated that, when the kidneys are preserved in a liquid formulation saturated with argon in accordance with the present invention, the architectural integrity of the kidney is preserved without any obvious glomerular modification, as shown in FIGS. 3A to 3D and 4A to 4E.

Moreover, like those represented in FIGS. 3A to 3D, histological observations of transverse histological sections of rat kidney (Tub denotes the renal tubule; Glom denotes the renal glomerulus) 14 days after transplantation show that:
in the air group, the kidneys exhibit subconfluent necrosis (FIG. 3B) and acute tubular necrosis (FIG. 3C) when compared with FIG. 3A which represents a section of normal kidney (control group),
in the argon group (FIG. 3D), the kidneys have an intact normal morphology as in the control group, with no modification nor any necrosis.

In addition, FIGS. 4A to 4E are reproductions of an immunohistochemistry, i.e. histological sections of rat kidneys, 14 days after transplantation, obtained for the various groups of rats, demonstrating that:
in the air group (FIG. 4A), the kidneys exhibit acute tubular necrosis with a complete loss of tubular and glomerular expression of CD10,
in the nitrogen group (FIG. 4B), the kidneys exhibit a significant expression of active caspase-3. Active caspase-3 is a marker for apoptosis (programmed cell death). High expression of active caspase-3 corresponds to induced apoptosis, leading to cell death and thus to an injured kidney,
in the xenon group (FIG. 4C), the active caspase-3 expression has been lost because of the serious acute tubular necrosis,
in the argon group, discrete and focal losses of CD 10 expression (FIG. 4D) and of active caspase-3 expression (FIG. 4E). CD10 is a protein in the brush border of the proximal tubule of the kidney. A weak CD10 expression corresponds to a damaged kidney.

The data obtained show the very positive and advantageous effects of argon on the preservation of a renal graft compared with the other experimental groups, i.e. the data obtained with nitrogen, air and xenon. There are also some good effects with xenon, but they are very much inferior to those obtained with argon.

Consequently, a liquid formulation comprising a liquid solution, such as the University of Wisconsin cold storage solution (UW) or the Celsior solution (CEL), and argon dissolved in said solution, can be successfully used to preserve and store biological materials, such as organs or other tissues which must be transplanted or grafted into an animal, preferably a mammal, in particular a human being.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1. A liquid formulation comprising a liquid solution and at least one gas selected from xenon, argon, hydrogen, H₂S, helium, krypton, neon, radon or CO, said gas being dissolved in said liquid solution, wherein the liquid formulation is adapted to be suitable for use as a preservation solution for preserving a biological material, the adaptation comprising the concentration of gas dissolved in the liquid formulation, expressed as a molar fraction, being from 0.1×10⁻⁴ to 4×10⁻⁴.

2. The liquid formulation of claim 1, wherein the gas is argon.

3. The liquid formulation of claim 1, wherein said biological material is chosen from biological cells, tissues and organs.

4. The liquid formulation of claim 1, wherein said biological material is a human material.

5. The liquid formulation of claim 1, wherein said biological material is an organ selected from the heart, the kidney, the liver, the pancreas and the intestines.

6. The liquid formulation of claim 1, wherein said biological material is a biological tissue or biological cells selected from bones, the bone marrow, tendons, the cornea, the heart valves, the veins, the arms, stem cells and the skin.

7. The liquid formulation of claim 1, wherein said liquid solution comprises water and at least one other substance selected from buffers, colloidal substances, impermeability agents, buffers, electrolytes, ROS eliminators and adenosine.

8. The liquid formulation of claim 1, wherein the liquid formulation comprises a dissolved gas concentration, expressed as a molar fraction, of from 0.1×10⁻⁴ to 0.5×10⁻⁴, preferably from 0.3×10⁻⁴ to 0.5×10⁻⁴.

9. The liquid formulation of claim 1, wherein said biological material is a human organ to be transplanted.

10. A method for preserving a biological material, in which the biological material to be preserved is brought into contact with a liquid formulation comprising a liquid solution and at least one gas selected from xenon, argon, hydrogen, H₂S, helium, krypton, neon, radon or CO, said gas being dissolved in said liquid solution, wherein the liquid formulation is adapted to be suitable for use as a preservation solution for preserving a biological material, the adaptation comprising the concentration of gas dissolved in the liquid formulation, expressed as a molar fraction, being from 0.1×10⁻⁴ to 4×10⁻⁴.

11. The method of claim 10, terized in thatwherein the liquid formulation is at a temperature of between 2° C. and 37° C., preferably less than 15° C., more preferably less than 10° C.

12. The method of claim 10, wherein said biological material is selected from the heart, the kidney, the liver, the pancreas and the intestines.

13. The method of claim 10, wherein the biological material is placed in a receptacle and in that the biological material is at least partially immersed in the liquid formulation.

14. The method of claim 13, wherein the receptacle comprises the liquid formulation, the biological material to be preserved and a gaseous atmosphere, said gaseous atmosphere comprising the gas(es) dissolved in the liquid formulation.

15. The method of claim 10, wherein the gas is argon.
